(19) United States
(12) Patent Application Publication
(10) Pub. No.: US 2026/0020875 A1
(43) Pub. Date: Jan. 22, 2026

(54) ULTRASONIC TREATMENT TOOL

(71) Applicant: OLYMPUS MEDICAL SYSTEMS CORP., Tokyo (JP)

(72) Inventor: Tsunetaka Akagane, Tokyo (JP)

(21) Appl. No.: 19/008,229

(22) Filed: Jan. 2, 2025

Related U.S. Application Data
(63) Continuation of application No. PCT/JP2024/025704, filed on Jul. 17, 2024.

(51) Int. Cl. A61B 17/32; A61B 17/00

(52) U.S. Cl. CPC A61B 17/320092; A61B 2017/00477; A61B 2017/320075; A61B 2017/320078; A61B 2017/320089

(57) ABSTRACT

An ultrasonic treatment tool includes: a tubular portion; an ultrasonic treatment unit provided on a distal end side of the tubular portion, the ultrasonic treatment unit including an ultrasonic transducer configured to generate ultrasonic vibration, and an ultrasonic blade that is connected to a distal end portion of the ultrasonic transducer and that includes a treatment portion configured to apply the ultrasonic vibration to a biological tissue to treat the biological tissue; and a support configured to accommodate the ultrasonic treatment unit inside the support with the treatment portion protruding from an inside toward an outside of the support, and support the ultrasonic treatment unit at a first node position of vibration of the ultrasonic transducer and at a second node position of vibration of the ultrasonic blade in the support when each of the ultrasonic transducer and the ultrasonic blade vibrates at a predetermined resonance frequency.

### CROSS REFERENCES TO RELATED APPLICATIONS

This application is a continuation of International Application No. PCT/JP2024/025704, filed on Jul. 17, 2024, the entire contents of which are incorporated herein by reference.

### BACKGROUND

### 1. Technical Field

The present disclosure relates to an ultrasonic treatment tool.

### 2. Related Art

In the related art, there has been known an ultrasonic treatment tool that treats a site to be treated (hereinafter, referred to as a treatment target) in a biological tissue by applying ultrasonic vibration to the treatment target (see, for example, US 2023/0240702 A).

In the ultrasonic treatment tool described in US 2023/0240702 A, an ultrasonic treatment unit is provided on a distal end side of a tubular portion inserted into a subject. The ultrasonic treatment unit includes an ultrasonic transducer that generates ultrasonic vibration, and an ultrasonic blade that is connected to a distal end of the ultrasonic transducer and includes a treatment portion that applies the ultrasonic vibration generated by the ultrasonic transducer to the treatment target.

In a general ultrasonic treatment tool, an ultrasonic treatment unit in which an ultrasonic transducer is provided on a proximal end side of a tubular portion, an ultrasonic blade is inserted into the tubular portion, and a treatment portion protrudes to the outside from a distal end of the tubular portion is adopted. That is, the ultrasonic treatment tool described in US 2023/0240702 A employs the ultrasonic treatment unit that is smaller than the ultrasonic treatment unit used in the general ultrasonic treatment tool.

### SUMMARY

In some embodiments, an ultrasonic treatment tool includes: a tubular portion configured to be inserted into a subject; an ultrasonic treatment unit provided on a distal end side of the tubular portion, the ultrasonic treatment unit including an ultrasonic transducer configured to generate ultrasonic vibration, and an ultrasonic blade that is connected to a distal end portion of the ultrasonic transducer and that includes a treatment portion configured to apply the ultrasonic vibration generated by the ultrasonic transducer to a biological tissue to treat the biological tissue; and a support configured to accommodate the ultrasonic treatment unit inside the support with the treatment portion protruding from an inside toward an outside of the support, and support the ultrasonic treatment unit at a first node position of vibration of the ultrasonic transducer and at a second node position of vibration of the ultrasonic blade in the support when each of the ultrasonic transducer and the ultrasonic blade vibrates at a predetermined resonance frequency.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating an ultrasonic treatment tool according to an embodiment;

FIG. 2 is a view for describing a configuration of an ultrasonic treatment unit;

FIG. 3 is a view for describing a configuration of the ultrasonic treatment unit;

FIG. 4 is a diagram for describing a structure for increasing an amplitude in the ultrasonic treatment unit;

FIG. 5 is a diagram for describing the structure for increasing the amplitude in the ultrasonic treatment unit;

FIG. 6 is a diagram for describing the structure for increasing the amplitude in the ultrasonic treatment unit;

FIG. 7 is a diagram for describing the structure for increasing the amplitude in the ultrasonic treatment unit;

FIG. 8 is a diagram for describing the structure for increasing the amplitude in the ultrasonic treatment unit;

FIG. 9 is a diagram for describing the structure for increasing the amplitude in the ultrasonic treatment unit;

FIG. 10 is a diagram for describing the structure for increasing the amplitude in the ultrasonic treatment unit; and

FIG. 11 is a view illustrating a modified example of the exemplary embodiment.

### DETAILED DESCRIPTION

Hereinafter, embodiments will be described with reference to the drawings. Note that the disclosure is not limited to the embodiments described below. Further, in the description of the drawings, the same reference signs denote the same parts.

### Schematic Configuration of Ultrasonic Treatment Tool

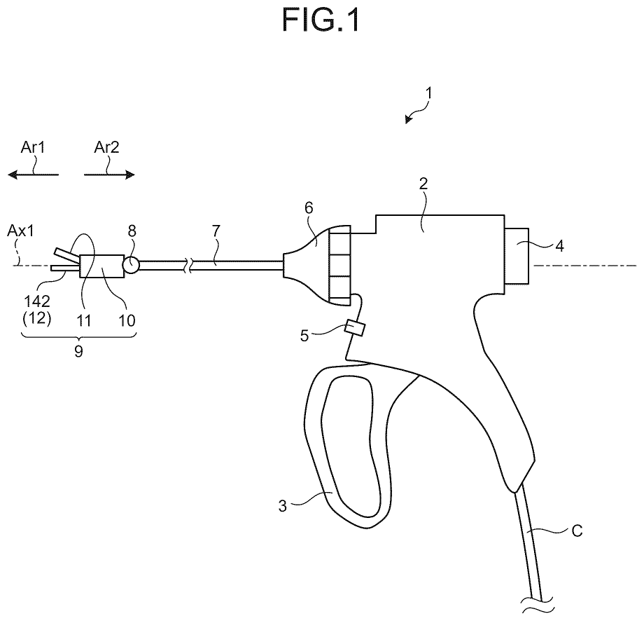

FIG. 1 is a view illustrating an ultrasonic treatment tool 1 according to an embodiment.

Hereinafter, one side along a central axis Ax1 of a sheath 7 is referred to as a distal end side Ar1, and the other side is referred to as a proximal end side Ar2.

The ultrasonic treatment tool 1 treats a site to be treated (hereinafter, referred to as a treatment target) in a biological tissue by applying treatment energy to the treatment target. The treatment energy in the present embodiment is ultrasonic energy and radio frequency energy. In addition, the treatment that can be performed by the ultrasonic treatment tool 1 according to the present embodiment is treatment such as coagulation (sealing) of the treatment target, incision of the treatment target, or the like. The coagulation and incision may be performed simultaneously. The treatment energy applied to the treatment target is not limited to both the ultrasonic energy and the radio frequency energy, and may be only the ultrasonic energy.

As illustrated in FIG. 1, the ultrasonic treatment tool 1 includes a holding case 2, an operation handle 3, a bending operating unit 4, a switch 5, a rotary knob 6, the sheath 7, a bent portion 8, and an end effector 9.

The holding case 2 supports the entire ultrasonic treatment tool 1.

The operation handle 3 is movably attached to the holding case 2 and receives an opening and closing operation by an operator such as a practitioner.

The bending operating unit 4 is rotatably provided in a state of being exposed to the outside from a side surface of the proximal end side Ar2 of the holding case 2, and receives a bending operation by the operator such as the practitioner.

The switch 5 is provided in a state of being exposed to the outside from a side surface of the distal end side Ar1 of the holding case 2, and receives a treatment operation by the operator such as the practitioner.

The rotary knob 6 has a substantially cylindrical shape coaxial with the central axis Ax1, and is provided on the distal end side Ar1 of the holding case 2. Then, the rotary knob 6 receives a rotation operation by the operator such as the practitioner. By the rotation operation, the rotary knob 6 rotates about the central axis Ax1 with respect to the holding case 2. As the rotary knob 6 rotates, the sheath 7, the bent portion 8, and the end effector 9 rotate about the central axis Ax1.

The sheath 7 is a cylindrical pipe and corresponds to a tubular portion. An end portion of the sheath 7 on the proximal end side Ar2 is inserted into the rotary knob 6 and fixed to an inner surface of the rotary knob 6.

The bent portion 8 is provided at an end portion of the sheath 7 on the distal end side Ar1, and bendably connects the end effector 9 to the sheath 7. That is, the end effector 9 is bent with respect to the sheath 7 when a bending mechanism (not illustrated) operates according to the bending operation of the bending operating unit 4 by the operator such as the practitioner. The bending mechanism (not illustrated) is configured using, for example, a wire, a rod, or the like, and connects the bending operating unit 4 and the end effector 9 to each other through the inside of the holding case 2 and the inside of the sheath 7.

The end effector 9 is connected to an end portion of the bent portion 8 on the distal end side Ar1 and treats the treatment target. As illustrated in FIG. 1, the end effector 9 includes a support 10, a jaw 11, and an ultrasonic treatment unit 12.

The support 10 is a member that supports the jaw 11 and the ultrasonic treatment unit 12 while being connected to the end portion of the bent portion 8 on the distal end side Ar1. The support 10 includes a tubular body in which an end portion of the support 10 on the proximal end side Ar2 is connected to the end portion of the bent portion 8 on the distal end side Ar1. Note that a support structure of the ultrasonic treatment unit 12 in the support 10 will be described in “Support Structure of Ultrasonic Treatment Unit in Support” described below.

At least a part of the jaw 11 is made of an electrically conductive material, and the jaw 11 is rotatably and axially supported with respect to an end portion of the support 10 on the distal end side Ar1. Then, the jaw 11 rotates with respect to the end portion of the support 10 on the distal end side Ar1 when an opening and closing mechanism (not illustrated) operates according to the opening and closing operation of the operation handle 3 by the operator such as the practitioner. With the rotation, the jaw 11 is opened and closed with respect to a treatment portion 142 provided at an end portion of the ultrasonic treatment unit 12 on the distal end side Ar1. Here, when the jaw 11 is closed with respect to the treatment portion 142, the treatment target is gripped between the jaw 11 and the treatment portion 142.

The ultrasonic treatment unit 12 generates ultrasonic vibration under the control of an external control device (not illustrated). The ultrasonic treatment unit 12 is inserted into the support 10, and is supported by the support 10 in a state where the treatment portion 142 protrudes from the distal end side Ar1 to the outside of the support 10.

Note that a detailed configuration of the ultrasonic treatment unit 12 will be described in “Configuration of Ultrasonic Treatment Unit” described below.

Then, the external control device (not illustrated) detects the treatment operation of the switch 5 by the operator such as the practitioner via an electric cable C. In addition, in a case where the treatment operation is detected, the control device applies the treatment energy to the treatment target gripped between the jaw 11 and the treatment portion 142 via the electric cable C.

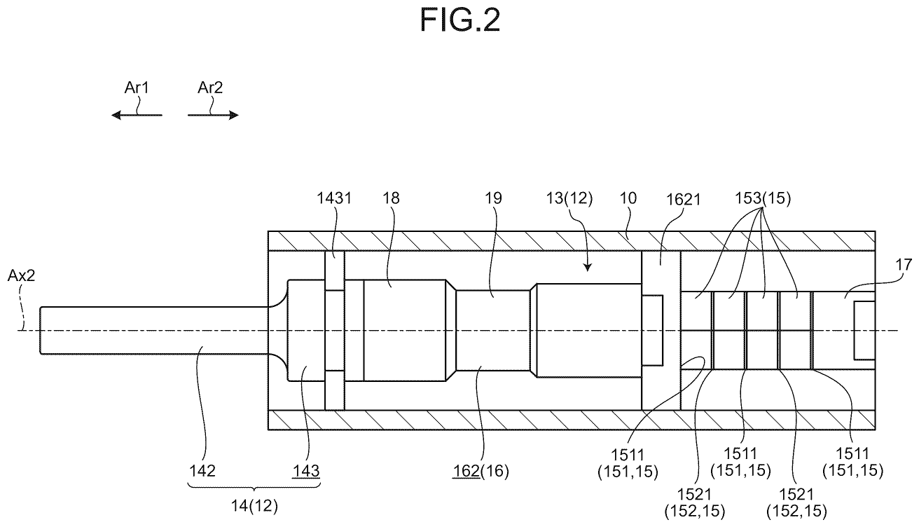

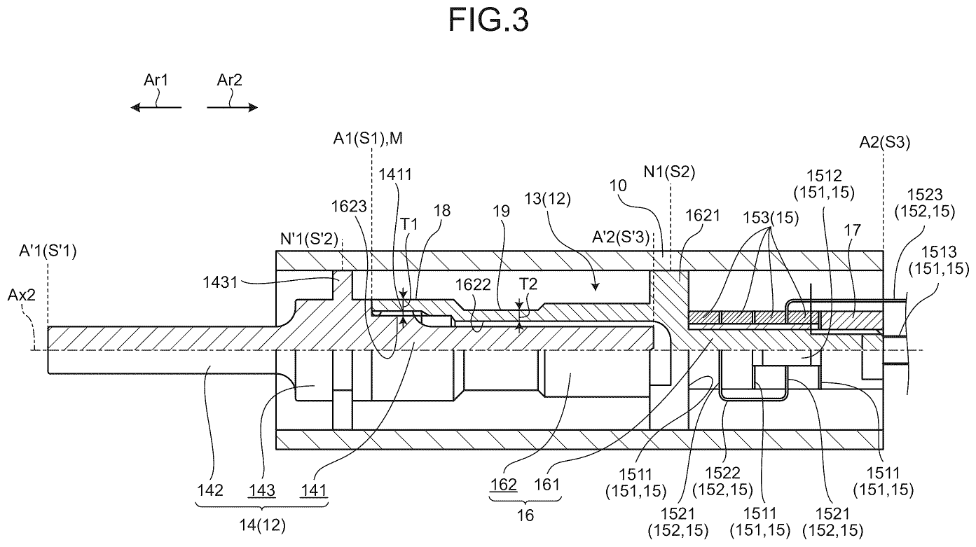

For example, when applying the ultrasonic energy to the treatment target, the external control device (not illustrated) supplies driving power to a piezoelectric element unit 15 described below in the ultrasonic treatment unit 12 via the electric cable C. As a result, the piezoelectric element unit 15 generates longitudinal vibration (ultrasonic vibration) in a direction along a central axis Ax2 (see FIGS. 2 and 3) of the support 10. In addition, the treatment portion 142 vibrates with a desired amplitude by the longitudinal vibration. Then, the ultrasonic vibration is applied from the treatment portion 142 to the treatment target gripped between the jaw 11 and the treatment portion 142. In other words, the ultrasonic energy is applied from the treatment portion 142 to the treatment target.

In addition, for example, when applying the radio frequency energy to the treatment target, the external control device (not illustrated) supplies radio frequency power between the jaw 11 and the ultrasonic treatment unit 12 via the electric cable C. When the radio frequency power is supplied between the jaw 11 and the ultrasonic treatment unit 12, a radio frequency current is supplied to the treatment target positioned between the jaw 11 and the treatment portion 142. In other words, the radio frequency energy is applied to the treatment target.

### Configuration of Ultrasonic Treatment Unit

Next, a configuration of the ultrasonic treatment unit 12 will be described.

FIGS. 2 and 3 are views illustrating the configuration of the ultrasonic treatment unit 12. Specifically, FIG. 2 is a cut-away view illustrating the inside of the support 10 along a plane including the central axis Ax2. FIG. 3 is a partially cut-away view illustrating the ultrasonic treatment unit 12 along the plane including the central axis Ax2 in FIG. 2.

As illustrated in FIGS. 2 and 3, the ultrasonic treatment unit 12 includes an ultrasonic transducer 13 and an ultrasonic blade 14.

The ultrasonic transducer 13 is a part that generates the ultrasonic vibration. As illustrated in FIGS. 2 and 3, the ultrasonic transducer 13 includes the piezoelectric element unit 15 and an element holding portion 16.

As illustrated in FIG. 3, the piezoelectric element unit 15 includes first and second electrode plates 151 and 152 and a plurality of (four in the present embodiment) piezoelectric elements 153.

The first and second electrode plates 151 and 152 are parts to which driving power is supplied from the external control device (not illustrated) via the electric cable C.

As illustrated in FIG. 3, the first electrode plate 151 includes a plurality of (three in the present embodiment) negative electrode plates 1511, a plurality of (two in the present embodiment) negative electrode wiring portions 1512, and a negative electrode terminal 1513. In FIG. 3, one of the two negative electrode wiring portions 1512 is not illustrated because the negative electrode wiring portion 1512 is positioned at a hidden position.

The plurality of negative electrode plates 1511 are each formed of an annular plate body, and are arranged side by side along the central axis Ax2.

The plurality of negative electrode wiring parts 1512 are portions that electrically connect outer edge portions of the negative electrode plates 1511 adjacent to each other.

The negative electrode terminal 1513 extends from an outer edge of the negative electrode plate 1511 positioned closest to the proximal end side Ar2 among the plurality of negative electrode plates 1511 toward the proximal end side Ar2. The negative electrode terminal 1513 is electrically connected to the external control device (not illustrated) via the electric cable C.

As illustrated in FIG. 3, the second electrode plate 152 includes a plurality of (two in the present embodiment) positive electrode plates 1521, a positive electrode wiring portion 1522 (one positive electrode wiring portion 1522 in the present embodiment), and a positive electrode terminal 1523.

The plurality of positive electrode plates 1521 are each formed of an annular plate body, and are arranged side by side along the central axis Ax2. The positive electrode plate 1521 has substantially the same shape as the negative electrode plate 1511. As illustrated in FIG. 3, the negative electrode plate 1511 and the positive electrode plate 1521 are alternately arranged along the central axis Ax2.

The positive electrode wiring portion 1522 is a part that electrically connects outer edge portions of the positive electrode plates 1521 adjacent to each other.

The positive electrode terminal 1523 extends from an outer edge of the positive electrode plate 1521 positioned closest to the proximal end side Ar2 among the plurality of positive electrode plates 1521 toward the proximal end side Ar2. In addition, the positive electrode terminal 1523 is electrically connected to the external control device (not illustrated) via the electric cable C. Then, the driving power is supplied from the external control device (not illustrated) between the negative electrode terminal 1513 and the positive electrode terminal 1523 via the electric cable C.

The plurality of piezoelectric elements 153 are each formed of an annular plate body and are arranged between the negative electrode plates 1511 and the positive electrode plates 1521. That is, the plurality of piezoelectric elements 153 are stacked along the central axis Ax2. Then, in the plurality of piezoelectric elements 153, a potential difference is generated in a stacking direction along the central axis Ax2 according to the driving power supplied to the first and second electrode plates 151 and 152, so that piezoelectric characteristics are generated, and displacement is alternately repeated in the stacking direction. As a result, the piezoelectric element unit 15 generates the ultrasonic vibration as the longitudinal vibration with the stacking direction as a vibration direction.

The element holding portion 16 is a member at least a partially made of an electrically conductive material, and as illustrated in FIG. 3, an element mounting portion 161 and a blade mounting portion 162 are integrally formed and hold the piezoelectric element unit 15.

The element mounting portion 161 is a bolt extending linearly along the central axis Ax2, and is inserted into each of the plurality of negative electrode plates 1511, the plurality of positive electrode plates 1521, and the plurality of piezoelectric elements 153. Then, as illustrated in FIG. 3, a fastening portion 17 which is a nut is attached to an end portion of the element mounting portion 161 on the proximal end side Ar2.

As illustrated in FIG. 3, the blade mounting portion 162 is provided at an end portion of the element mounting portion 161 on the distal end side Ar1, and has a substantially cylindrical shape extending linearly toward the distal end side Ar1 along the central axis Ax2. The blade mounting portion 162 is set to have a larger diameter than the element mounting portion 161. Therefore, the plurality of negative electrode plates 1511, the plurality of positive electrode plates 1521, and the plurality of piezoelectric elements 153 are sandwiched between the blade mounting portion 162 and the fastening portion 17 in a state where the element mounting portion 161 penetrates along the central axis Ax2, so that they are integrally fastened in a state of having a substantially cylindrical shape. That is, the ultrasonic transducer 13 is implemented by a bolt-clamped Langevin-type transducer.

In the blade mounting portion 162, as illustrated in FIG. 3, a first flange portion 1621 having a larger outer diameter than other portions is provided at an end portion of the blade mounting portion 162 on the proximal end side Ar2.

As illustrated in FIG. 3, an insertion recess 1622 is provided in the blade mounting portion 162. The insertion recess 1622 is a recess linearly extending from an end surface of the blade mounting portion 162 on the distal end side Ar1 toward the proximal end side Ar2 along the central axis Ax2. A screw portion 1623 functioning as a female screw is provided on a side surface of the insertion recess 1622 at an end portion on the distal end side Ar1. In the ultrasonic transducer 13, a part at which the screw portion 1623 is provided corresponds to a connection portion 18. The connection portion 18 includes the end surface of the blade mounting portion 162 on the distal end side Ar1.

The ultrasonic blade 14 is connected to the connection portion 18 and applies the ultrasonic vibration generated by the ultrasonic transducer 13 to the treatment target. As illustrated in FIG. 3, the ultrasonic blade 14 is a member in which a protrusion 141 and the treatment portion 142 are integrally formed with an intermediate portion 143 interposed therebetween.

The intermediate portion 143 is a cylindrical member extending linearly along the central axis Ax2. As illustrated in FIG. 3, a second flange portion 1431 having a larger outer diameter than other portions is provided in the intermediate portion 143.

The protrusion 141 is a cylindrical member having an outer diameter set smaller than that of the intermediate portion 143 and linearly extending from an end surface of the intermediate portion 143 on the proximal end side Ar2 toward the proximal end side Ar2 along the central axis Ax2. Then, the protrusion 141 is inserted into the insertion recess 1622. A screw portion 1411 functioning as a male screw is provided on an outer circumferential surface of the protrusion 141 at an end portion on the distal end side Ar1 as illustrated in FIG. 3. Then, the ultrasonic blade 14 is connected to the ultrasonic transducer 13 by screwing the screw portion 1411 into the screw portion 1623. In this state, a portion of the protrusion 141 other than the screw portion 1411 is not in contact with an inner surface of the insertion recess 1622. In addition, the end surface of the blade mounting portion 162 on the distal end side Ar1 abuts on the end surface of the intermediate portion 143 on the proximal end side Ar2.

The treatment portion 142 extends from an end surface of the intermediate portion 143 on the distal end side Ar1 toward the distal end side Ar1 along the central axis Ax2. Then, the treatment portion 142 applies the ultrasonic vibration generated by the ultrasonic transducer 13 to the treatment target.

In the ultrasonic treatment unit 12 according to the present embodiment, the amplitude of the ultrasonic vibration generated by the piezoelectric element unit 15 is increased as described below.

Structure for Increasing Amplitude in Ultrasonic Treatment Unit

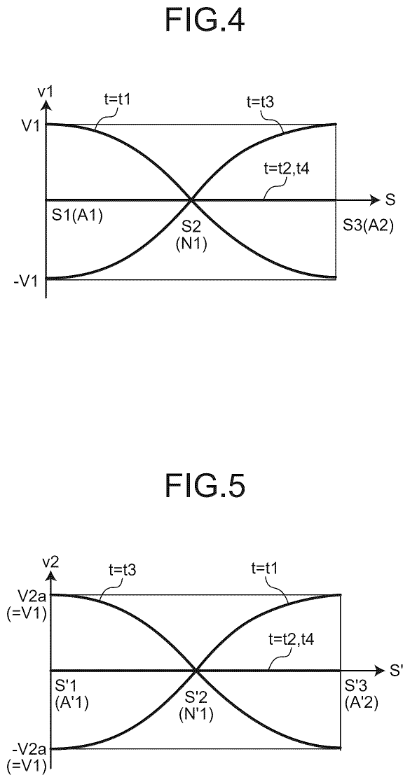

FIGS. 4 to 10 are diagrams for describing a structure for increasing the amplitude in the ultrasonic treatment unit 12.

When the piezoelectric element unit 15 generates the ultrasonic vibration, the element holding portion 16 undergoes the ultrasonic vibration as the longitudinal vibration with a direction along the central axis Ax2 as the vibration direction at a predetermined resonance frequency f0 (hereinafter, referred to as first vibration).

FIG. 4 is a diagram illustrating a change in the first vibration (v1) with respect to a change in a position (S) in a longitudinal direction along the central axis Ax2 in a case where the element holding portion 16 vibrates alone. Note that FIG. 4 illustrates the first vibration (v1) at times t=t1, t2, t3, and t4.

Then, in the first vibration at the predetermined resonance frequency f0, a position S1 at which the end surface of the blade mounting portion 162 on the distal end side Ar1 (a distal end of the element holding portion 16) is positioned is a first antinode position A1 as illustrated in FIGS. 3 and 4. In the first vibration, a position S3 at which an end surface of the element mounting portion 161 on the proximal end side Ar2 (a proximal end of the element holding portion 16) is positioned is a first antinode position A2. In the element holding portion 16, a position of the distal end corresponds to the first antinode position A1 and a position of the proximal end corresponds to the first antinode position A2, and thus, the element holding portion 16 can vibrate at the predetermined resonance frequency f0. A position S2 positioned at an intermediate position between the position S1 and the position S3 is a first node position N1 of the first vibration. That is, the ultrasonic transducer 13 has only one node position of vibration in a state of vibrating at the predetermined resonance frequency f0.

Here, an amplitude of the first vibration at each position (S) of the element holding portion 16 along the central axis Ax2 is defined as a first amplitude. In the first vibration of the element holding portion 16 alone, the first amplitude at the first antinode positions A1 and A2 has a magnitude V1 as illustrated in FIG. 4.

In addition, as the first vibration is transmitted from the element holding portion 16, the ultrasonic blade 14 undergoes the ultrasonic vibration as the longitudinal vibration with the direction along the central axis Ax2 as the vibration direction at the predetermined resonance frequency f0 that is identical to that of the first vibration (hereinafter, referred to as second vibration).

FIG. 5 is a diagram illustrating a change in the second vibration with respect to a change in a position (S′) in the longitudinal direction along the central axis Ax2 in a case where the ultrasonic blade 14 vibrates alone. Note that FIG. 5 illustrates the second vibration (v2) at times t=t1, t2, t3, and t4.

Then, in the second vibration at the predetermined resonance frequency f0, a position S′1 at which an end surface of the treatment portion 142 on the distal end side Ar1 (a distal end of the ultrasonic blade 14) is positioned is a second antinode position A′1 as illustrated in FIGS. 3 and 5. Further, in the second vibration, a position S′3 at which an end surface of the protrusion 141 on the proximal end side Ar2 (a proximal end of the ultrasonic blade 14) is positioned is a second antinode position A′2. In the ultrasonic blade 14, a position of the distal end corresponds to the second antinode position A′1 and a position of the proximal end corresponds to the second antinode position A′2, and thus, the ultrasonic blade 14 can vibrate at the predetermined resonance frequency f0. Then, a position S′2 positioned at an intermediate position between the position S′1 and the position S′3 is a second node position N′1 of the second vibration. That is, the ultrasonic blade 14 has only one node position of vibration in a state of vibrating at the predetermined resonance frequency f0.

Here, an amplitude of the second vibration at each position (S′) of the ultrasonic blade 14 along the central axis Ax2 is defined as a second amplitude. In the second vibration of the ultrasonic blade 14 alone, the second amplitude at the second antinode positions A′1 and A′2 has a magnitude V2a as illustrated in FIG. 5. The magnitude V2a of the second amplitude is the same as the magnitude V1 of the first amplitude described above.

In the ultrasonic blade 14, the end surface of the intermediate portion 143 on the proximal end side Ar2 that abuts on the end surface of the blade mounting portion 162 on the distal end side Ar1 is positioned at an intermediate position M different from the second antinode positions A′1 and A′2 and the second node position N′1 of the second vibration. In the present embodiment, the intermediate position M is positioned between the second node position N′1 and the second antinode position A′2 of the second vibration, and is positioned at the first antinode position A1 of the first vibration (a position of the end surface of the blade mounting portion 162 on the distal end side Ar1). That is, the connection portion 18 is provided at a position including the antinode position (first antinode position A1) of the vibration of the ultrasonic transducer 13 and at a position away from the antinode position (second antinode position A′2) of the vibration of the ultrasonic blade 14 in a state of vibrating at the predetermined resonance frequency f0.

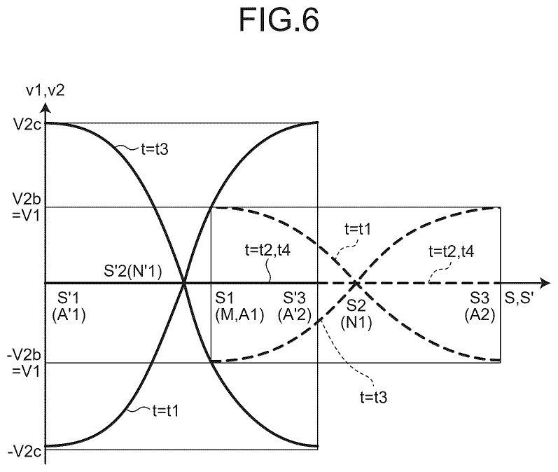

FIG. 6 is a diagram illustrating the changes in the first and second vibrations with respect to the changes in the positions (S and S′) in the longitudinal direction along the central axis Ax2 in a case where the ultrasonic treatment unit 12 vibrates. Note that FIG. 6 illustrates the first vibration (v1) and the second vibration (v2) at times t=t1, t2, t3, and t4. In FIG. 6, the first vibration of the element holding portion 16 is indicated by a broken line, and the second vibration of the ultrasonic blade 14 is indicated by a solid line.

As illustrated in FIGS. 3 and 6, also in a case where the ultrasonic treatment unit 12 vibrates at the predetermined resonance frequency f0, the element holding portion 16 undergoes the first vibration at the predetermined resonance frequency f0 similarly to a case where the element holding portion 16 undergoes the first vibration alone. Therefore, the first antinode positions A1 and A2 and the first node position N1 in a direction parallel to the central axis Ax2 are the same as the first antinode positions A1 and A2 and the first node position N1 when the element holding portion 16 undergoes the first vibration alone, respectively. In addition, also in a case where the ultrasonic treatment unit 12 vibrates at the predetermined resonance frequency f0, the first amplitude at the first antinode positions A1 and A2 has the magnitude V1, similarly to a case where the element holding portion 16 undergoes the first vibration alone.

As illustrated in FIGS. 3 and 6, also in a case where the ultrasonic treatment unit 12 vibrates at the predetermined resonance frequency f0, the ultrasonic blade 14 undergoes the second vibration at the predetermined resonance frequency f0, similarly to a case where the ultrasonic blade 14 undergoes the second vibration alone. Therefore, the second antinode positions A′1 and A′2 and the second node position N′1 in the direction parallel to the central axis Ax2 are the same as the second antinode positions A′1 and A′2 and the second node position N′1 when the ultrasonic blade 14 undergoes the second vibration alone, respectively.

Here, in the ultrasonic treatment unit 12, the ultrasonic vibration is transmitted from the element holding portion 16 to the ultrasonic blade 14 via the end surface of the blade mounting portion 162 on the distal end side Ar1 and the end surface of the intermediate portion 143 on the proximal end side Ar2 that abut on each other. The end surface of the blade mounting portion 162 on the distal end side Ar1 is positioned at the first antinode position A1 of the first vibration. In addition, the end surface of the intermediate portion 143 on the proximal end side Ar2 that abuts on the end surface of the blade mounting portion 162 on the distal end side Ar1 is positioned at the intermediate position M of the second vibration. The first antinode position A1 and the intermediate position M are the same position.

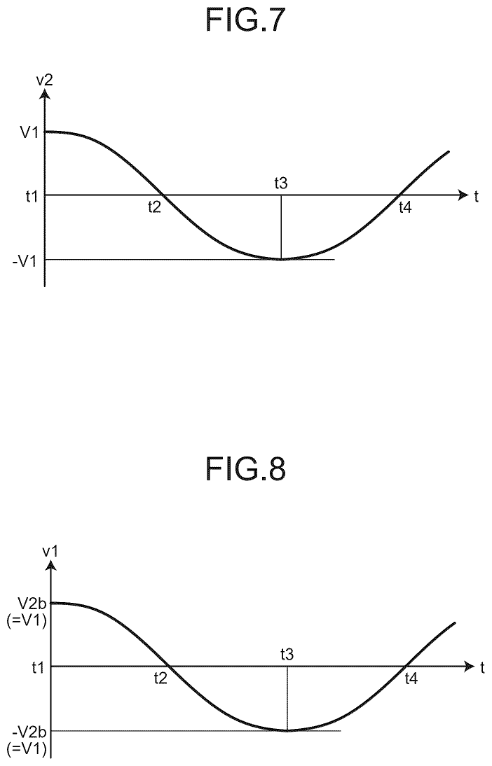

FIG. 7 is a diagram illustrating a temporal change in the first vibration at the first antinode position A1 in a case where the ultrasonic treatment unit 12 vibrates at the predetermined resonance frequency f0. FIG. 8 is a diagram illustrating a temporal change in the second vibration at the intermediate position M in a case where the ultrasonic treatment unit 12 vibrates at the predetermined resonance frequency f0.

As illustrated in FIGS. 6 to 8, in a case where the ultrasonic treatment unit 12 vibrates, the first amplitude of the first vibration at the first antinode position A1 has the magnitude V1. The second amplitude of the second vibration at the intermediate position M has a magnitude V2b.

Here, the first antinode position A1 and the intermediate position M are the same position. Therefore, the magnitude V2b of the second amplitude at the intermediate position M is the same as the magnitude V1 of the first amplitude at the first antinode position A1. The first vibration at the first antinode position A1 and the second vibration at the intermediate position M are in the same phase with each other.

In a case where the ultrasonic treatment unit 12 vibrates, the magnitude V2b of the second amplitude at the intermediate position M different from the second antinode positions A′1 and A′2 is the same as the magnitude V1 of the first amplitude at the first antinode positions A1 and A2 of the first vibration. Then, in the second vibration, the second amplitude at the second antinode positions A′1 and A′2 is larger than the second amplitude at the intermediate position M. Therefore, in a case where the ultrasonic treatment unit 12 vibrates at the predetermined resonance frequency f0, a magnitude V2c of the second amplitude at the second antinode positions A′1 and A′2 is larger than the magnitude V1 of the first amplitude at the first antinode positions A1 and A2 of the first vibration.

As described above, in the present embodiment, it is possible to increase the second amplitude of the ultrasonic vibration at the second antinode position A′1 positioned at the distal end of the ultrasonic blade 14 without providing a cross-sectional area change portion (horn) whose cross-sectional area perpendicular to the central axis Ax2 is changing along the central axis Ax2.

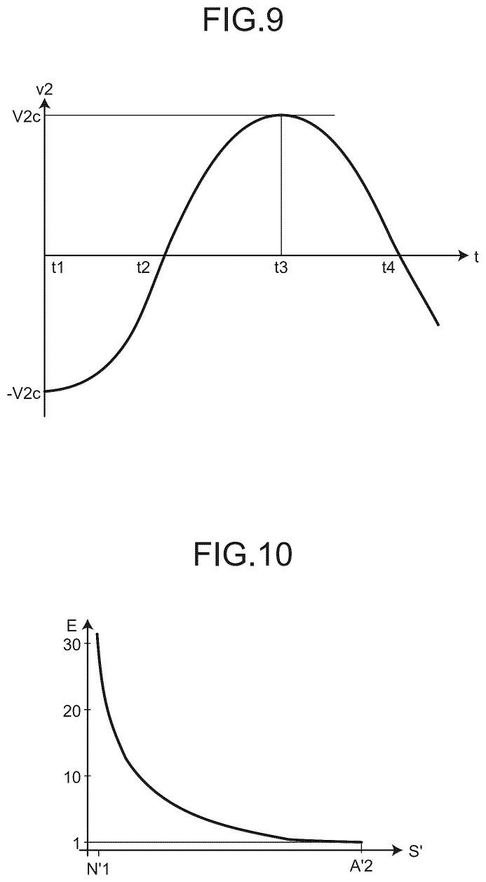

FIG. 9 is a diagram illustrating a temporal change in the second vibration at the second antinode position A′1 in the ultrasonic blade 14 in a case where the ultrasonic treatment unit 12 vibrates.

As described above, the second antinode position A′1 is positioned at a distal end of the ultrasonic blade 14. As illustrated in FIG. 9, the magnitude V2c of the second amplitude at the second antinode position A′1 is larger than the magnitude V1 of the first amplitude at the first antinode position A1. In the second vibration, the intermediate position M is positioned between the second node position N′1 and the second antinode position A′2. In this case, the second vibration at the second antinode position A′1 is opposite phase to the first vibration at the first antinode position A1.

FIG. 10 illustrates an increase ratio (E) of the second amplitude at the second antinode positions A′1 and A′2 of the second vibration with respect to the first amplitude at the first antinode positions A1 and A2 of the first vibration in a case of changing the intermediate position M along the central axis Ax2 between the second node position N′1 and the second antinode position A′2 in the ultrasonic blade 14.

As described above, the magnitude V2b of the second amplitude at the intermediate position M is the same as the magnitude V1 of the first amplitude at the first antinode positions A1 and A2. Therefore, the magnitude V2b of the second amplitude at the intermediate position M is the same as the magnitude V1 of the first amplitude at the first antinode positions A1 and A2 regardless of a position change in the intermediate position M along the central axis Ax2. That is, the magnitude V2b of the second amplitude at the intermediate position M does not change according to the position change in the intermediate position M along the central axis Ax2.

Meanwhile, as the intermediate position M approaches the second node position N′1, a ratio E of the magnitude V2c of the second amplitude at the second antinode positions A′1 and A′2 with respect to the magnitude V2b of the second amplitude at the intermediate position M increases. Since the magnitude V1 of the first amplitude at the first antinode position A1 is the same as the magnitude V2b of the second amplitude at the intermediate position M, as the intermediate position M approaches the second node position N′1, the increase ratio E of the magnitude V2c of the second amplitude at the second antinode positions A′1 and A′2 with respect to the magnitude V1 of the first amplitude at the first antinode positions A1 and A2 increases as illustrated in FIG. 10.

### Connection Strength Between Ultrasonic Transducer and Ultrasonic Blade

Next, a connection strength between the ultrasonic transducer 13 and the ultrasonic blade 14 will be described with reference to FIG. 3.

In the present embodiment, the ultrasonic treatment unit 12 is provided on the distal end side Ar1 of the sheath 7 as described above. That is, the ultrasonic treatment unit 12 is smaller than an ultrasonic treatment unit used in a general ultrasonic treatment tool. In a case where such a small ultrasonic treatment unit 12 is adopted as described above, there is a possibility that the connection strength between the ultrasonic transducer 13 and the ultrasonic blade 14 becomes weak.

Therefore, in the present embodiment, a thickness T1 of the connection portion 18 is set to be larger than a thickness T2 of a part 19 of the blade mounting portion 162 that is adjacent to the connection portion 18 as illustrated in FIG. 3.

### Support Structure of Ultrasonic Treatment Unit in Support

Next, a support structure of the ultrasonic treatment unit 12 in the support 10 will be described with reference to FIG. 3.

As illustrated in FIG. 3, the first flange portion 1621 is provided at the first node position N1 of the first vibration. In addition, the second flange portion 1431 is provided at the second node position N′1 of the second vibration.

The support 10 supports the ultrasonic treatment unit 12 on an inner surface of the support 10 by the first and second flange portions 1621 and 1431. As a result, the ultrasonic treatment unit 12 is prevented from being bent due to a force applied at the time of treatment of the treatment target, and is prevented from moving along the central axis Ax2 and rotating about the central axis Ax2. The support 10 may directly support the first and second flange portions 1621 and 1431, or may employ a configuration in which another member such as rubber is interposed between each of the first and second flange portions 1621 and 1431 and the support 10.

Here, the supporting at the first node position N1 by the support 10 is fixed at least in a direction perpendicular to a longitudinal axis (central axis Ax2) of the ultrasonic treatment tool 1. Further, the supporting is not fixed in a rotation direction of the longitudinal axis. Furthermore, the supporting is not fixed in a direction of the longitudinal axis.

In addition, the supporting at the second node position N′1 by the support 10 is fixed at least in a direction perpendicular to the longitudinal axis (central axis Ax2) of the ultrasonic treatment tool 1. Further, the supporting is fixed in the rotation direction of the longitudinal axis. Furthermore, the supporting is fixed in the direction of the longitudinal axis.

According to the first embodiment described above, the following effects are obtained.

In the ultrasonic treatment tool 1 according to the present embodiment, the support 10 supports the ultrasonic treatment unit 12, which is housed therein, at the first node position N1 and the second node position N′1 in the support 10. That is, the support 10 can support the ultrasonic treatment unit 12 at two positions (first and second positions N1 and N′1) within one wavelength of vibration, and can stably receive a force in a bending direction of the ultrasonic blade 14.

Therefore, with the ultrasonic treatment tool 1 according to the present embodiment, the ultrasonic blade 14 can be stably supported, and it is possible to appropriately cope with the size reduction of the ultrasonic treatment unit 12.

Furthermore, in the ultrasonic treatment tool 1 according to the present embodiment, the ultrasonic transducer 13 has only one node position of vibration in a state of vibrating at the predetermined resonance frequency f0. In addition, the ultrasonic transducer 13 has a distal end and a proximal end at antinode positions of vibration. Therefore, the ultrasonic treatment unit 12 can be reduced in size.

In the ultrasonic treatment tool 1 according to the present embodiment, the ultrasonic blade 14 has only one node position of vibration in a state of vibrating at the predetermined resonance frequency f0. In addition, the ultrasonic blade 14 has a distal end and a proximal end at antinode positions of vibration. Therefore, the ultrasonic treatment unit 12 can be reduced in size.

Furthermore, in the ultrasonic treatment tool 1 according to the present embodiment, the connection portion 18 is provided at a position including the antinode position of vibration of the ultrasonic transducer 13 and at a position away from the antinode position of vibration of the ultrasonic blade 14 in a state where each of the ultrasonic transducer 13 and the ultrasonic blade 14 vibrates at the predetermined resonance frequency. Therefore, it is possible to increase the second amplitude of the ultrasonic vibration at the second antinode position A′1 positioned at the distal end of the ultrasonic blade 14 without providing the cross-sectional area change portion (horn) whose cross-sectional area perpendicular to the central axis Ax2 is changing along the central axis Ax2. That is, since it is not necessary to provide the horn, the ultrasonic treatment unit 12 can be reduced in size.

### Other Embodiments

Although the embodiment has been described so far, the disclosure should not be limited only to the above-described embodiment.

In the above-described embodiment, the end effector 9 is bendably connected to the sheath 7 by the bent portion 8, but the disclosure is not limited thereto, and a configuration in which the end effector 9 is directly fixed to the sheath 7, that is, a configuration in which the end effector 9 cannot be bent with respect to the sheath 7, may be adopted.

In the above-described embodiment, the ultrasonic transducer 13 and the ultrasonic blade 14 each have only one node position of vibration in a state of vibrating at the predetermined resonance frequency f0, but the disclosure is not limited thereto, and the ultrasonic transducer 13 and the ultrasonic blade 14 may each have two or more node positions.

In the above-described embodiment, a configuration in which the jaw 11 is omitted may be adopted.

In the above-described embodiment, the protrusion 141 is provided in the ultrasonic blade 14, and the insertion recess 1622 is provided in the ultrasonic transducer 13, but the disclosure is not limited thereto. Conversely, a configuration in which the protrusion 141 is provided in the ultrasonic transducer 13, and the insertion recess 1622 is provided in the ultrasonic blade 14 may be adopted.

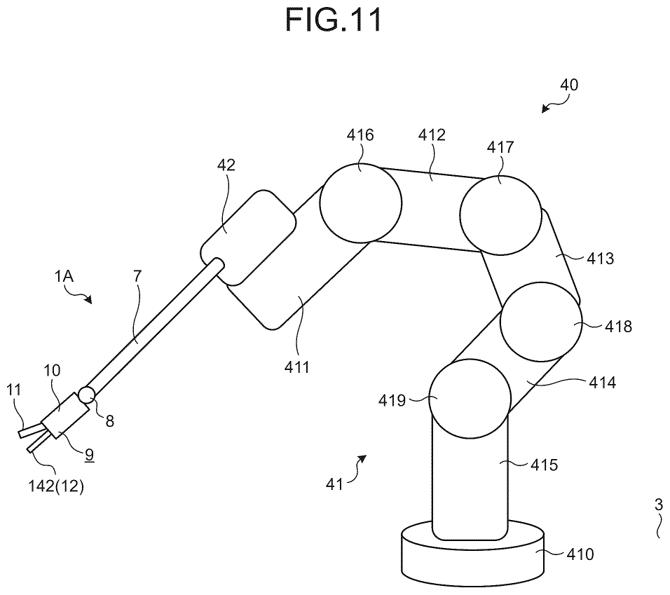

FIG. 11 is a view illustrating a modified example of the embodiment.

In the above-described embodiment, the ultrasonic treatment tool is implemented by the handheld ultrasonic treatment tool 1, but the embodiment is not limited thereto. For example, as in the present modified example illustrated in FIG. 11, the ultrasonic treatment tool may be adopted in a medical apparatus 40 including a robot arm 41. Hereinafter, an ultrasonic treatment tool according to the present modified example will be referred to as an ultrasonic treatment tool 1A for convenience of description.

As illustrated in FIG. 11, the robot arm 41 includes a base portion 410, first to fifth arm portions 411 to 415, and first to fourth joint portions 416 to 419.

The base portion 410 is installed on a floor surface or the like and supports the entire medical apparatus 40.

The first to fifth arm portions 411 to 415 are connected in series by the first to fourth joint portions 416 to 419. Among the first to fifth arm portions 411 to 415, the fifth arm portion 415 positioned on a proximal end side is fixed on the base portion 410. In addition, the ultrasonic treatment tool 1A is detachably connected to the first arm portion 411 positioned on a distal end side among the first to fifth arm portions 411 to 415.

The first to fourth joint portions 416 to 419 relatively rotate a pair of mutually connected arm portions among the first to fifth arm portions 411 to 415 about mutually different axes. That is, in the present modified example, the ultrasonic treatment tool 1A is movable in four degrees of freedom. Note that the robot arm 41 is not limited to having four degrees of freedom, and may have another different number of degrees of freedom. That is, the number of first to fifth arm portions 411 to 415 and the number of first to fourth joint portions 416 to 419 are not limited to the above-described numbers, and may be other numbers.

Although not specifically illustrated, actuators for relatively rotating a pair of mutually connected arm portions among the first to fifth arm portions 411 to 415 are provided inside the first to fourth joint portions 416 to 419. Each actuator is driven under the control of the external control device (not illustrated).

As illustrated in FIG. 11, the ultrasonic treatment tool 1A includes a detachable portion 42 in addition to the sheath 7, the bent portion 8, and the end effector 9 described in the above-described embodiment.

The detachable portion 42 is a part that is provided at a proximal end of the sheath 7 and attaches and detaches the ultrasonic treatment tool 1A to and from the robot arm 41 (first arm portion 411). Although not specifically illustrated, an actuator for performing an opening and closing operation of the jaw 11 with respect to the treatment portion 142 and a bending operation of the end effector 9 with respect to the sheath 7 under the control of the external control device (not illustrated) is provided inside the detachable portion 42.

Even in a case where the ultrasonic treatment tool according to the disclosure is adopted in the medical apparatus 40 including the robot arm 41 as in the present modified example described above, the same effects as those of the above-described embodiment are obtained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool comprising:
a tubular portion configured to be inserted into a subject;
an ultrasonic treatment unit provided on a distal end side of the tubular portion, the ultrasonic treatment unit including
an ultrasonic transducer configured to generate ultrasonic vibration, and
an ultrasonic blade that is connected to a distal end portion of the ultrasonic transducer and that includes a treatment portion configured to apply the ultrasonic vibration generated by the ultrasonic transducer to a biological tissue to treat the biological tissue; and
a support configured to
accommodate the ultrasonic treatment unit inside the support with the treatment portion protruding from an inside toward an outside of the support, and
support the ultrasonic treatment unit at a first node position of vibration of the ultrasonic transducer and at a second node position of vibration of the ultrasonic blade in the support when each of the ultrasonic transducer and the ultrasonic blade vibrates at a predetermined resonance frequency.

2. The ultrasonic treatment tool according to claim 1, wherein the supporting at the first node position of the vibration of the ultrasonic transducer by the support is fixed at least in a direction perpendicular to a longitudinal axis of the ultrasonic treatment tool.

3. The ultrasonic treatment tool according to claim 2, wherein the supporting at the first node position of the vibration of the ultrasonic transducer by the support is not fixed in a rotation direction of the longitudinal axis of the ultrasonic treatment tool.

4. The ultrasonic treatment tool according to claim 3, wherein the supporting at the first node position of the vibration of the ultrasonic transducer by the support is not fixed in a direction of the longitudinal axis of the ultrasonic treatment tool.

5. The ultrasonic treatment tool according to claim 2, wherein the supporting at the second node position of the vibration of the ultrasonic blade by the support is fixed at least in the direction perpendicular to the longitudinal axis of the ultrasonic treatment tool.

6. The ultrasonic treatment tool according to claim 5, wherein the supporting at the second node position of the vibration of the ultrasonic blade by the support is fixed in a rotation direction of the longitudinal axis of the ultrasonic treatment tool.

7. The ultrasonic treatment tool according to claim 6, wherein the supporting at the second node position of the vibration of the ultrasonic blade by the support is fixed in a direction of the longitudinal axis of the ultrasonic treatment tool.

8. The ultrasonic treatment tool according to claim 1, wherein
the ultrasonic blade includes the treatment portion and a protrusion protruding toward the ultrasonic transducer, and
an insertion recess into which the protrusion is inserted is provided in the ultrasonic transducer.

9. The ultrasonic treatment tool according to claim 1, wherein the ultrasonic transducer has only one first node position.

10. The ultrasonic treatment tool according to claim 1, wherein the ultrasonic blade has only one second node position.

11. The ultrasonic treatment tool according to claim 1, wherein a connection portion of the ultrasonic transducer with respect to the ultrasonic blade is provided at a position including an antinode position of vibration of the ultrasonic transducer and a position away from an antinode position of vibration of the ultrasonic blade when each of the ultrasonic transducer and the ultrasonic blade vibrates at the resonance frequency.

12. The ultrasonic treatment tool according to claim 1, wherein the ultrasonic transducer has a distal end and a proximal end at antinode positions of vibration when vibrating at the resonance frequency.

13. The ultrasonic treatment tool according to claim 1, wherein the ultrasonic blade has a distal end and a proximal end at antinode positions of vibration when vibrating at the resonance frequency.

14. The ultrasonic treatment tool according to claim 1, wherein
a first flange portion supported by the support is provided at the first node position in the ultrasonic transducer, and
a second flange portion supported by the support is provided at the second node position in the ultrasonic blade.

15. The ultrasonic treatment tool according to claim 1, further comprising a bent portion provided at a distal end of the tubular portion and configured to bendably connect the ultrasonic treatment unit to the tubular portion.

16. The ultrasonic treatment tool according to claim 1, further comprising a jaw that is openable and closable with respect to the treatment portion and is configured to grip the biological tissue between the treatment portion and the jaw.

17. The ultrasonic treatment tool according to claim 1, further comprising a handle provided on a proximal end side of the tubular portion and configured to be operated by an operator.

18. The ultrasonic treatment tool according to claim 1, wherein the ultrasonic treatment tool is connected to a distal end of a robot arm.
